(19) United States
(12) Patent Application Publication
(10) Pub. No.: US 2010/0158997 A1
(43) Pub. Date: Jun. 24, 2010

(54) BLOW-MOLDED THIN-WALLED DRUG DELIVERY CAPSULES

(76) Inventor: Liang Chang DONG, Sunnyvale, CA (US)

Correspondence Address: MORRISON & FOERSTER LLP, 12531 HIGH BLUFF DRIVE, SUITE 100, SAN DIEGO, CA, 92130-2040, US

(21) Appl. No.: 12/639,873

(22) Filed: Dec. 16, 2009

Related U.S. Application Data
(60) Provisional application No. 61/138,844, filed on Dec. 18, 2008.

(51) Int. Cl. A61K 9/52; A61P 43/00

(52) U.S. Cl. 424/452

(57) ABSTRACT

A thin-walled capsule of defined permeability is produced by blow-molding an aqueous-based polymer composition.

### CROSS-REFERENCE TO RELATED APPLICATIONS

This application claims priority from U.S. provisional application 61/138,844 filed 18 Dec. 2008. The contents of this document are incorporated herein by reference.

### TECHNICAL FIELD

The invention is directed to injection, blow-molded capsules for controlled release of therapeutic or other beneficial agents in vivo or in other aqueous environments. The agent is released through an orifice by virtue of the presence of an osmotic push layer.

### BACKGROUND ART

A large number of designs for dispensing drugs using osmotic push layers surrounded by a semi-permeable membrane that allows the aqueous surroundings to expand this layer and eject the drug have been disclosed. Such disclosures appear, for example, in U.S. Pat. Nos. 4,892,778; 4,940,465; 4,915,949 and more recently U.S. Pat. No. 6,183,466; U.S. Pat. No. 6,551,613; U.S. Pat. No. 6,929,803 and U.S. Pat. No. 7,147,867. A generic view of this type of system is shown in FIG. 1 which represents the state of the prior art in general. As seen in FIG. 1, a semi-permeable coating encompasses an osmotic push compartment and a drug compartment. As water enters the osmotic push compartment, the drug is expelled through an orifice/exit port at one end of the capsule.

While this general concept is present in the art, techniques for making appropriate capsules with appropriate permeability qualities have not been perfected. In many cases, for example, such as that described in U.S. Pat. No. 7,147,867 (supra), the preparation methods employ organic solvents to form the polymeric-based wall of the capsule. In other embodiments, such as those set forth in U.S. Pat. No. 6,551,613, an internal cap section and a space between the drug-containing layer and the external wall are required. U.S. Pat. No. 6,596,314 describes capsules that have double-layered walls.

The use of organic solvents in the process of preparing the outer walls of the capsules is avoided by the techniques described in U.S. Pat. No. 5,614,578 which employ polymer compositions containing polycaprolactone and polyethylene glycol as a flux enhancer. Using these compositions, also, permeabilities in the range of 10⁻³-10⁻⁵ in units of cm mil/hour·atm were achieved. A related patent, U.S. Pat. No. 5,830,502, discloses capsules that are formed by injection molding.

U.S. Pat. No. 6,153,678 also discloses polymeric materials for the formation of capsule walls using injection molding. The capsule is formed from caprolactone, polyalkalene oxides and polyoxyethylenated fatty acids or esters.

In the methods described in these patents, a high-injection pressure and temperature are required to extrude the viscous polymeric melt through a thin-walled mold conduit, often causing phase-separation of components in the formed membranes. Such phase-separation may result in erratic and uncontrollable water-permeability of the resulting membrane.

### DISCLOSURE OF THE INVENTION

The invention is directed to a thin-walled capsule of controlled water-permeability which encloses as an osmotic delivery system. Because the capsule wall is thin and provides consistent permeability in a defined range, effective delivery of beneficial agents at a desired rate over a prolonged period of time can be achieved.

Thus, in one aspect, the invention is directed to a blow-molded capsule comprising a release rate-controlling polymer membrane having a water-permeability of 10⁻⁷-10⁻³ cm²/hour·atm, wherein said membrane is 0.1-0.5 mm in thickness. In use, the capsule is provided with a “drug layer” (which may contain any beneficial agent(s) the release of which is desired) and an osmotic push layer and has an orifice/exit port proximal to the drug layer.

In another aspect, the invention is directed to a method to deliver a drug or other beneficial agent in vivo or to other aqueous environments employing the capsules of the invention. Thus, the capsules may be administered orally to animals, or may be used ex vivo, for example, to release a pesticide in an agricultural setting.

In another aspect, the invention is directed to a method to prepare a capsule having the desired characteristics which method comprises blow-molding a preformed thick-membrane capsule.

### BRIEF DESCRIPTION OF THE DRAWINGS

FIG. 1 shows a generic osmotic delivery capsule as is known in the art.

FIG. 2 shows the invention capsule encasement.

### MODES OF CARRYING OUT THE INVENTION

In one aspect, the invention provides capsule encasements for controlled release oral dosage forms that are osmotic delivery systems with various desired release profiles including a Constant rate, Ascending rate, Spatial delivery and Temporal delivery (CAST) by employing injection blow-molding of appropriate compositions. An orally administered capsule can achieve delivery over a prolonged period of time, for example, at least over 15 minutes, 1 hour, 2 hours or 4 hours or more. The delivery may be at a constant rate over this time period, or the rate of delivery may increase during this time.

The variable aspects described above are mediated by factors known in the art involving the osmotic push layer and the beneficial agent or drug layer contained within the delivery device. Although the permeability of the wall has an influence on the rate of delivery in general, whether the rate of delivery is constant or ascending depends on the design of the osmotic push layer, wherein the pattern of expansion of the osmotic push layer controls whether release is constant or ascending. Time dependence is readily controlled by design of the drug layer whereby the drug layer may have, for example, a portion composed of placebo closest to the exit port so that the drug or other beneficial compound is not released immediately. Similarly, the spatial aspect of release—i.e., the location, for example, in the digestive tract at which an oral composition releases drug can be controlled by the effect of pH, for example, on the swelling of the osmotic layer. All of these features can be at least partially regulated by appropriate design of the osmotic push layer and the drug layer independent of the capsule encasement of the invention.

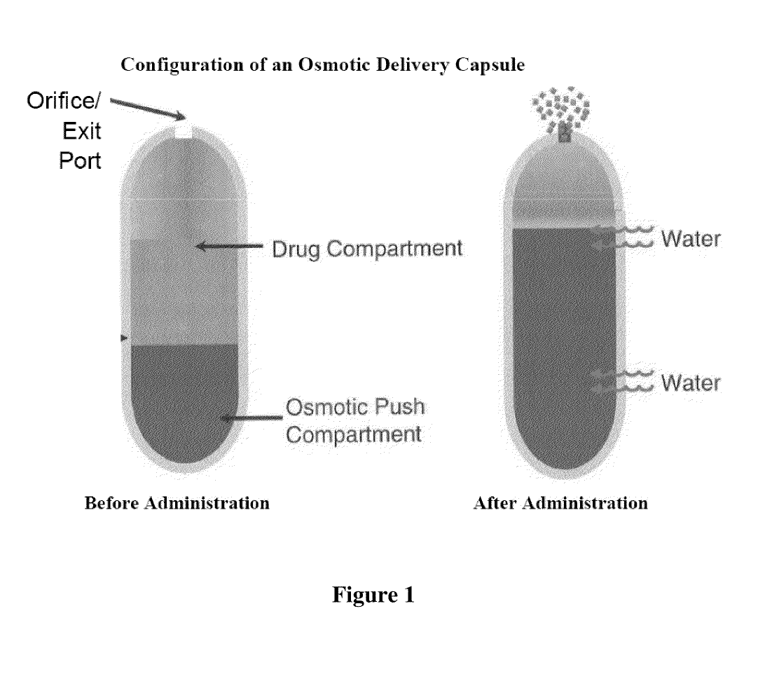

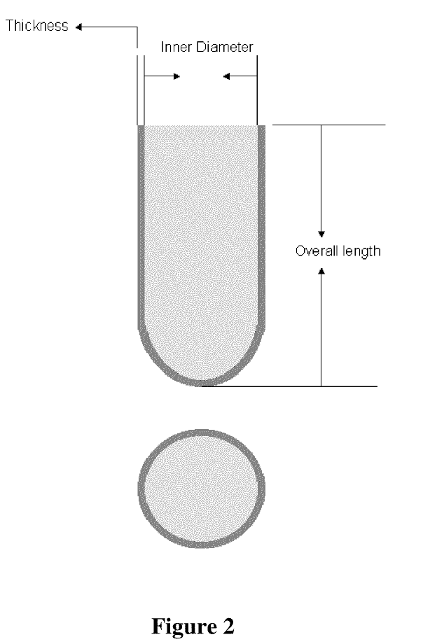

However, in general, the rate of release is also related to the permeability of the wall of the capsule to water in its surroundings. The surroundings may be an in vivo or other aqueous environment, such as a food product to be preserved. The permeability can be controlled by the thickness of the wall and by its composition. The “wall” of the capsule or the “membrane” or the “encasement” of the capsule are used herein interchangeably. This refers to the outer barrier surrounding a drug layer and an osmotic push layer as shown in FIG. 1, and shown empty in FIG. 2. As also shown in FIG. 1, the capsules of the invention may be provided with an orifice adjacent the drug layer which can be formed by heat crimping the outer membrane over the drug layer.

It is important that the encasement have a controlled permeability. Permeability is defined as the rate of transit of a given volume over time multiplied by the membrane thickness divided by the osmotic pressure used in the test and by the permeation area. The units used in the present case are cm²/hour·atmospheres. The thin-walled capsules of the invention can achieve a controlled permeability within the range of 10⁻³-10⁻⁷ or 10⁻³-10⁻⁵ in these units. This is as compared to the permeabilities of about 10⁻⁶-10⁻⁹ in the same units in the U.S. Pat. No. 5,614,578 patent, supra.

In the process of forming the thin-walled capsules of the invention, the following steps are taken:

A polymeric composition is first extruded through a screw assembly, where the polymer composition melts, or is melted by other means. Melting may precede blending the composition. The molten polymer composition is injected, for example, through a nozzle into a hollow, heated capsule preform mold containing a core rod. The preform mold opens and the core rod is rotated and clamped into a hollow, chilled blow mold. The core rod opens and allows compressed air into the preformed capsule in the chilled blow mold, which inflates it to the configuration of a thin-wall capsule. After a cooling period, the blow mold opens, and the finished thin-wall capsule is stripped off of the core rod. The preform mold and blow mold each can have many cavities to meet the required output.

The injection blow-molding process thus involves two main steps: first, forming of the preform capsule with a relatively thicker wall and second, blow-molding of the preform capsule into a final thin-wall capsule. This two-step process assures the homogeneous composition of finished thin-wall capsules with consistent water-permeability.

The materials employed in the formation of the blow-molded capsules of the invention are polymers that are blended without the use of organic solvents. Such polymers can be melted and blended or otherwise mixed without employing organic solvents.

The injection blow-molded thin wall capsule serves as a release rate-controlling membrane, in which a variety of cores comprising a therapeutic agent can be incorporated. Therefore, for in vivo use, the delivery system can be designed to match circadian rhythm for chronotherapy, to target biopharmaceutical formulations to different sections of gastrointestinal tract (targeting a vaccine formulation to the Peyer's patches at the lower small intestine, targeting monoclonal antibody to the lower GI tract for inflammatory bowel disease or a protein/peptide formulation to the colon for enhanced oral absorption), and to provide versatile release profiles as well.

The delivery platform of the invention offers advantages over existing osmotic delivery systems, that involve use of organic solvents, since the process for preparation is organic solvent-free. Furthermore, the injection blow-molded capsules of the invention are composed of materials that are bioerodable.

Primary polymer materials for injection blow-molding of the invention capsules include, but are not limited to, polycaprolactone polymers with molecular weight between 10,000 and 100,000 and acrylic polymers (Eudragit® RS/Eudragit® RS/Eudragit® NE) with molecular weight between 10,000 and 1,000,000. The molecular weight of the polycaprolactone polymers strongly impacts their melt viscosity, thereby their injection-moldability. To maneuver the melt viscosity, blends of high and low molecular weight polycaprolactones can be prepared to provide a desired melt viscosity for injection blow-molding. Secondary polymer materials are hydrophilic polymers, either water-soluble or water-swellable. Representative polymers are poly(oxyethylene-co-oxypropylene), hydroxypropylcellulose, acrylic polymers (Eudragit® L100-55, Eudragit® L100), and other water-soluble/water swellable celluloses. These polymers serve as water permeation enhancers to render the injection blow-molded capsules with suitable water permeability.

The terms “beneficial agent”, “therapeutic agent” and “drug” are used interchangeably herein, and they refer to an agent, drug compound, composition of matter or mixture thereof which provides either a therapeutic effect or an effect that is otherwise beneficial. Thus “beneficial agent” includes not only drugs, but pesticides, herbicides, germicides, biocides, algaecides, rodenticides, fungicides, insecticides, antioxidants, plant-growth promoters, plant growth inhibitors, preservatives, antipreservatives, disinfectants, sterilization agents, catalysts, chemical reactants, fermentation agents, foods, food supplements, nutrients, cosmetics, drugs, vitamins, sex sterilants, fertility inhibitors, fertility promoters, microorganism attenuators and other agents that benefit the environment of use. As used herein, drug or therapeutic beneficial agents include any physiologically or pharmacologically active substances that produce a localized or systemic effect or effects in animals, including warm blooded mammals, humans and primates; avians; domestic household or farm animals such as cats, dogs, sheep, goats, cattle, horses, and pigs; laboratory animals such as mice, rats and guinea pigs; fish; reptiles, zoo and wild animals; and the like. An active drug that can be delivered includes inorganic and organic compounds, including, without limitation, drugs which act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autocold systems, the alimentary and excretory systems, the histamine system, and the central nervous system. Suitable agents may be selected from, for example, proteins, enzymes, hormones, polynucleotides, nucleoproteins, polysaccharides, glycoproteins, lipoproteins, polypeptides, steroids, hypnotics and sedatives, psychic energizers, tranquilizers, anticonvulsants, muscle relaxants, anti-Parkinson agents, analgesics, anti-inflammatories, local anesthetics, muscle contractants, antimicrobials, antimalarials, hormonal agents including contraceptives, sympathomimetics, polypeptides and proteins capable of eliciting physiological responses diuretics, lipid regulating agents, antiandrogenic agents, antiparasitics, neoplastics, antineoplastics, hypoglycemics, nutritional agents and supplements, growth supplements, fats, ophthalmics, antienteritis agents, electrolytes and diagnostic agents.

Examples of beneficial agents for use in the capsules of the invention are venlafaxine hydrochloride, bupropion HCl, metoprolol succinate, prochlorperazine edisylate, ferrous sulfate, aminocaproic acid, mecaxylamine hydrochloride, procainamide hydrochloride, amphetamine sulfate, methamphetamine hydrochloride, benzphetamine hydrochloride, isoproteronol sulfate, phenmetrazine sulfate, isoproteronol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperazine maleate, anisindone, diphenadione, erythrityl tetranitrate, digoxin, isoflurophate, acetazolamide, methazolamide, bendroflumethiazide, chlorpropamide, tolazamide, chlormadinone acetate, phenaglycodol, allopurinol, aluminum aspirin, methotrexate, acetyl sulfisoxazole, erythromycin, hydrocortisone, hydrocorticosterone acetate, cortisone acetate, dexamethasone and its derivatives such as betamethasone, triamcinolone, methyltestosterone, 17-β-estradiol, ethinyl estradiol, ethinyl estradiol 3-methyl ether, pednisolone, 17-β-hydroxyprogesterone acetate, 19-norprogesterone, norgestrel, norethindrone, norethisterone, norethiederone, progesterone, norgesterone, norethisterone, norethynodrel, aspirin, indomethacin, naproxen, fenoprofen, sulindac, indoprofen, nitroglycerin, isosorbide dinitrate, propranolol, timolol, atenolol, alprenolol, cimetidine, clonidine, enitabas, imipramine, levodopa, chlorpromazine, methyldopa, dihydroxyphenylalanine, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, captropril, mandol, quabenz, hydrochlorothiazide, ranitidine, flurbiprofen, fenbufen, fluprofen, tolmetin, alclofenac, mefenamic, flufenamic, difuninal, nimodipine, nitrendipine, nisoldipine, nicardipine, felodipine, lidofiazine, tiapamil, gallopamil, amlodipine, mioflazine, lisinopril, enalapril, captopril, ramipril, endlapriat, famotidine, nizatidine, sucralfate, etindinine, tetratolol, minoxidil, chlordiazepoxide, diazepam, amitriptylin, and imipramine. Further examples are proteins and proteins which include, but are not limited to, insulin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, porcine somatropin, oxytocin, vasopressin, prolactin, somatostatin, lypressin, pancreozymin, luteinizing hormone, LHRH, interferons, interleukins, growth hormones such as human growth hormone, bovine growth hormone and porcine growth hormone, fertility inhibitors such as the prostaglandins, fertility promoters, growth factors, and human pancreas hormone releasing factor.

Two or more therapeutic or other beneficial agents can be incorporated into the capsules. Such agents can be in a wide variety of chemical and physical forms, such as uncharged molecules, components of molecular complexes, nonirritating pharmaceutically acceptable salts, derivatives of a therapeutic agent such as ethers, esters, amides, etc. Derivatives of therapeutic agents are easily hydrolyzed by the body pH and enzymes. For in vivo use, the amount of therapeutic agent(s) in the dosage form is an amount necessary to produce the desired response. In practice, this will vary widely depending upon the particular therapeutic agent(s), the site of delivery, the severity of the medical condition, and the desired therapeutic effect. Thus, often it is not practical to define a particular therapeutic range for a therapeutically effective dose of the therapeutic active agent incorporated into the dosage form, however, the dosage form generally will contain 0.1 mg to 1.0 g of a therapeutic agent.

The osmotic push composition contained in the invention capsules contains an expandable means such as an osmopolymer, hydrogel, or other expandable member that interacts with water, or aqueous biological fluids so as to expand to an equilibrium state, as well as an osmotic agent. Osmopolymers, for example, exhibit the ability to swell in water and retain a significant portion of the imbibed water within the polymer structure. The osmopolymers swell or expand to a very high degree, usually exhibiting a 2 to 50 fold volume increase. Osmopolymers can be noncrosslinked or crosslinked. The swellable, hydrophilic polymers are, in some embodiment, lightly crosslinked, containing, for example, cross-links formed by covalent or ionic bonds. Osmopolymers can be of plant, animal or synthetic origin. Hydrophilic polymers suitable for the present purpose include poly(hydroxyalkylmethacrylate); poly(vinylpyrrolidone); anionic and cationic hydrogels; polyelectrolyte complexes, poly(vinyl alcohol) having a low acetate residual, crosslinked with formaldehyde, or glutaraldehyde; a mixture of methyl cellulose, crosslinked agar and carboxymethyl cellulose, a water insoluble, water swellable copolymer produced by forming a dispersion of finely divided copolymer of maleic anhydride with styrene, ethylene, propylene, butylene or isobutylene crosslinked with from 0.0001 to about 0.5 moles of polyunsaturated crosslinking agent per mole of maleic anhydride in the copolymer; water swellable polymers of N-vinyl lactams, and the like.

Other osmopolymers include polymers that form hydrogels such as Carbopol® acidic carboxy polymers, the sodium salt of Carbopol® acidic carboxy polymers and other metal salts; Cyanamer® polyacrylamides; crosslinked water swellable indene maleic anhydride polymers; Goodrite® polyacrylic acid, and the sodium and other metal salts; Polyox® polyethylene oxide polymers; starch graft copolymers; Aqua-Keeps® acrylate polymers; diester crosslinked polyglucan, and the like. Representative polymers that form hydrogels are known in the art, for example, as disclosed in U.S. Pat. No. 3,865,108; U.S. Pat. No. 4,207,893, and in Handbook of Common Polymers, by Scott and Roff, published by the Chemical Rubber, CRC Press, Cleveland, Ohio.

Osmotic agents include various electrolytes, such as sodium chloride and/or saccharides such as glucose, sucrose or sorbitol.

The following examples are intended to illustrate but not to limit the invention.

### Example 1

### Preparation of Capsule Wall

A polymer composition is prepared as follows: 59.5 wt % poly(caprolactone), 25.5 wt % of poly(ethylene oxide) possessing a 5,000,000 molecular weight, and 15 wt % of poly(oxyethylene-co-oxypropylene) identified as Pluronic F-127 are blended at a temperature range of 65-95° C., using a mixer to produce a homogeneous blend. The blend is extruded at 80-90° C. and pelletized at 25° C., using an extruder and pelletizer. The pellets are injection-blow-molded into thin wall capsules, as shown in FIG. 2. The dimensions of the representative capsules are shown below.

| | Capsule size | | |
|---|---|---|---|
| | 00 | 0 | 1 |
| Overall length (mm) | 28.00 | 25.50 | 22.00 |
| Inner diameter (mm) | 8.25 | 7.40 | 6.70 |
| Thickness (mm) | 0.1-0.5 | 0.1-0.5 | 0.1-0.5 |

### Example 2

The procedure of Example 1 is followed using 63 wt % poly(caprolactone), 27 wt % poly(ethylene oxide), and 10 wt % Pluronic F-127.

### Example 3

The procedure of Example 1 is performed using 58 wt % poly(caprolactone), 32 wt % poly(ethylene oxide) and 10 wt % Pluronic F-127.

### Example 4

The procedure of Example 1 is performed using 67.9 wt % poly(caprolactone), 29.1 wt % poly(ethylene oxide) and 3 wt % Pluronic F-127.

### Example 5

The procedure of Example 1 is performed using 62.3 wt % poly(caprolactone), 27 wt % poly(ethylene oxide), 10 wt % Pluronic F-127, and 0.7 wt % of a member selected from the group consisting of lactose, or fructose, or Cab-o-Sil, a colloidal silicon dioxide as a nucleating agent.

### Example 6

The procedure of Example 1 is performed using 40-95 wt % poly(caprolactone), 5-60% poly(ethylene oxide), and 0-20 wt % poly(oxyethylene-co-oxypropylene).

### Example 7

The procedure of Example 1 is performed using 40-95 wt % poly(caprolactone), 5-60% hydroxypropylcellulose (Klucel™ EF), and 0-20 wt % poly(oxyethylene-co-oxypropylene).

### Example 8

The procedure of Example 1 is performed using 40-95 wt % acrylic polymers (Eudragit® RS/Eudragit® RS/Eudragit® NE), 5-60% an enteric acrylic polymer (Eudragit® L100--55).

### Example 9

### Preparation of Osmotic Delivery Capsules

A. Preparation of the Drug Layer Granulation:

A binder solution is prepared by adding hydroxypropylcellulose (“HPC”) to water in a ratio of 5 mg HPC to 0.995 g of water. The solution is mixed until HPC is dissolved. All the excipients except Mg stearate are first milled and screened. A fluid bed granulator (“FBG”) bowl is charged with the required amounts of the drug-layer formulation. After mixing the dry materials in the bowl, the binder solution prepared above is sprayed. The granulation is dried in the FBG until the target moisture content (<1% by weight water) is reached. The granulation is milled through a 7 mesh screen and transferred to a tote blender or a V-blender. The required amount of lubricant, Mg stearate is sized using a 40 mesh screen and are blended into the granulation using the tote or V-blender until uniformly dispersed (about 1 minute).

B. Preparation of the Osmotic-push Layer Granulation:

A binder solution is prepared by dissolving hydroxypropylcellulose (“HPC”) to water in a ratio of 5 mg HPC to 0.995 g of water. The solution is mixed until HPC is dissolved. All the excipients except Mg stearate are first milled and screened. A fluid bed granulator (“FBG”) bowl is charged with the required amounts of the osmotic-push formulation. After mixing the dry materials in the bowl, the binder solution prepared above is sprayed. The granulation is dried in the FBG until the target moisture content (<1% by weight water) is reached. The granulation is milled through a 7 mesh screen and transferred to a tote blender or a V-blender. The required amount of lubricant, Mg stearate is sized using a 40 mesh screen and are blended into the granulation using the tote or V-blender until uniformly dispersed (about 1 minute).

C. Bilayer Core Compression:

A longitudinal tablet press is set up with round, deep concave or modified ball punches and dies. The punch/dies are selected to assure the compressed tablets are conformed to the shape of the bottom of the injection-blow molded capsule. Two feed hoppers are placed on the press. The drug layer prepared as above is placed in one of the hoppers while the osmotic push layer prepared as above is placed in the remaining hopper. The initial adjustment of the tableting parameters is performed to produce cores with a uniform target drug layer weight. The second layer adjustment (osmotic push layer) of the tableting parameters is performed which bond the drug layer to the push layer to produce final bi-layer tablets with uniform weight, thickness, hardness and friability.

D. Assembling and Orifice Forming

An osmotic delivery system prepared as follows comprises an injection blow-molded capsule consisting of the poly(caprolactone), poly(ethylene oxide) and Pluronic F-127 composition that envelopes an internal space with an osmotic push layer at the closed bottom and a drug-pull layer at the opened mouth. The capsule wall is prepared as in Example 1. The push-layer comprises 58.75 wt % sodium carboxymethylcellulose, 30.00 wt % sodium chloride, 10.00 wt % hydroxypropylcellulose, 1.00 wt % red ferric oxide, and 0.25 wt % magnesium stearate. The drug-layer comprises 33.8 wt % of venlafaxine hydrochloride, 33.0 wt % of hydroxypropylcellulose, having an 80,000 average molecular weight, 32.7 wt % mannitol, and 0.5 wt % of magnesium stearate. The opened mouth of the dosage form is crimped by a heating means to form a 0.381 mm orifice. The delivery system contains venlafaxine hydrochloride equivalent to 37.5 mg, 75 mg or 150 mg venlafaxine free base for once-daily administration (QD). A different size capsule is used to accommodate the different doses.

### Example 10

The procedure of Example 9 is employed, but substituting a drug-layer that comprises 60.0 wt % of bupropion hydrochloride, 20.0 wt % of hydroxypropylcellulose, having 140,000 average molecular weight, 19.5 wt % mannitol, and 0.5 wt % of magnesium stearate. The delivery system contains 150 mg, or 300 mg bupropion hydrochloride for once-daily administration (QD).

### Example 11

The procedure of Example 9 is repeated but substituting drug-layer that comprises 38.0 wt % of metoprolol succinate, 30.0 wt % of hydroxypropylcellulose, having an 80,000 average molecular weight, 31.5 wt % mannitol, and 0.5 wt % of magnesium stearate. The delivery system contains 23.75 mg, 47.5 mg, 95 mg and 190 mg metoprolol succinate equivalent to 25 mg, 50 mg, 100 mg and 200 mg of metoprolol tartrate for once-daily administration (QD).

### Example 12

The procedure of Example 9 is repeated except that the osmotic push-layer comprises 55 wt % kappa-carrageenan, 28.0 wt % sorbitol, 15 wt % polyvinylpyrrolidone, 1.00 wt % red ferric oxide and 1.0 wt % stearic acid.

1. A blow-molded capsule comprising a release rate-controlling, polymeric membrane, having a water permeability of 10⁻⁷-10⁻³ in units of cm²/hr·atm wherein said membrane is 0.1-0.5 mm in thickness.

2. The capsule of claim 1 wherein the membrane is composed of polycaprolactone and/or acrylic polymers in combination with poly(ethylene oxide) and/or hydroxypropylcellulose and optionally poly(oxyethylene-co-oxypropylene) and/or poly(oxyethylene 40 stearate).

3. The capsule of claim 1 which is prepared by a method comprising
(a) blending said polymeric components in a molten state;
(b) injection molding the blend of step (a) to form a preform with a thick membrane; followed by:
(c) blow-molding the preform of step (b).

4. The capsule of claim 1 which further comprises a first layer comprising at least one beneficial agent in contact with and atop a second layer which comprises an osmotic push composition.

5. The capsule of claim 4 which further comprises a release orifice abutting the first layer.

6. The capsule of claim 5 wherein said release orifice is formed by heating and crimping an opened mouth of the capsule against the top of the first layer.

7. The capsule of claim 4 wherein the osmotic push composition is composed of an osmotic polymer and an osmotic agent.

8. The capsule of claim 7 wherein the osmotic polymer is poly(ethylene oxide) and/or sodium carboxymethylcellulose and/or kappa-carrageenan and the osmotic agent is sodium chloride and/or a saccharide.

9. A method to effect release of a beneficial agent into an environment which method comprises supplying said environment with the capsule of claim 4.

10. The method of claim 9 wherein said environment is the digestive tract of an animal.

11. A method to prepare a capsule for delivery of a beneficial agent using a push layer which method comprises blow-molding a preformed thick membrane capsule comprised of polymers prepared without organic solvents to obtain a blow molded capsule for said agent and layer.

12. The method of claim 11, wherein the preformed thick membrane is formed from a molten blend of said polymers released from a preform mold.
